(19) United States
(12) Patent Application Publication
(10) Pub. No.: US 2010/0035813 A1
(43) Pub. Date: Feb. 11, 2010

(54) RECOMBINANT E-SELECTIN MADE IN INSECT CELLS

(75) Inventors: Gale SMITH, Darnestown, MD (US); Peter Pushko, Frederick, MD (US); Vittoria Cioce, Kensington, MD (US)

Correspondence Address: COOLEY GODWARD KRONISH LLP;ATTN: Patent Group, Suite 1100, 777 - 6th Street, NW, WASHINGTON, DC, 20001, US

(73) Assignee: Novavax Inc.

(21) Appl. No.: 12/509,033

(22) Filed: Jul. 24, 2009

Related U.S. Application Data
(63) Continuation of application No. 11/369,788, filed on Mar. 7, 2006, now abandoned.
(60) Provisional application No. 60/660,258, filed on Mar. 10, 2005.

(51) Int. Cl. A61K 38/16; C07K 14/47; C07K 7/08; C12N 15/11; C12N 15/00; C12N 5/10; C12N 1/21; C12P 21/02

(52) U.S. Cl. 514/12; 530/350; 530/326; 536/23.5; 435/320.1; 435/325; 435/252.3; 435/348; 435/69.1

(57) ABSTRACT

The inventive features include recombinant mammalian E-selectin peptides, nucleic acids encoding said peptides, vectors and cells having these nucleic acids, and methods of making the peptides. Further inventive features include methods of treating diseases and conditions associated with inflammation using recombinant mammalian E-selectin peptides to induce mucosal tolerance to E-selectin.

### RELATED APPLICATIONS

This application is a Continuation of U.S. application Ser. No. 11/369,788, filed Mar. 7, 2006, which claims the benefit of U.S. Provisional Application No. 60/660,258, filed Mar. 10, 2005, each of which are hereby incorporated by reference in their entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: NOVV 025 02US SeqList_ST25.txt, date recorded: Jul. 24, 2009, file size 23 kilobytes).

### BACKGROUND OF THE INVENTION

E-selectin is a cell surface glycoprotein cell adhesion molecule that is cytokine inducible and is found exclusively on endothelial cells. E-selectin mediates the adhesion of various leukocytes, including neutrophils, monocytes, eosinophils, natural killer (NK) cells and a subset of T cells, to activated endothelium. The expression of E-selectin is induced on human endothelial cells in response to the inflammation associated cytokines of IL-1 and TNF alpha, as well as to lipopolysaccharide (LPS), through transcriptional upregulation.

Conventional anti-inflammatory interventions include depleting the circulatory pool of leukocytes, inhibiting leukocyte function, and using immunosuppressive drugs such as cyclosporine A and FK506. However, most available immunosuppressive agents have systemic side effects that limit their long term use.

Mucosal administration of autoantigens has been shown to suppress inflammation and disease activity in models of stroke and arteriosclerosis as well as in several models of autoimmunity such as diabetes, arthritis, and experimental allergic encephalomyelitis. Administration of multiple low doses of E-selectin via nasal/oral administration induced mucosal tolerance to E-selectin. Mucosal tolerance is a well established model whereby immunological tolerance is induced to a specific antigen through nasal instillation or feeding of that antigen. Antigen administered nasally encounters nasally associated lymphoid tissue which has evolved to protect the host from invading pathogens and developed the inherent property of preventing the host from reacting to inhaled proteins that are not pathogenic. Active tolerance with production of regulatory T cells occurs after repetitive administrations of low-dose antigen.

E selectin expression is not constitutive, being virtually limited to endothelium that is becoming activated in response to inflammatory stimuli, such as IL-1, TNF-alpha, or LPS. E-selectin may be chronically expressed at the site of local inflammation in vivo, and as such E-selectin serves as an appropriate tolerizing molecule to guide regulatory T cells that have been tolerized to E-selectin to local sites of endothelial activation. These regulatory T cells that have been tolerized with a low-dose regimen secrete cytokines such as IL-10 and transforming growth factor (TGF) b1 on antigen restimulation which suppress TH1 immune responses. Although activation of these T cells is specific for the tolerizing antigen (in this case E-selectin), the immunomodulatory cytokines secreted in response to activation have non-specific effects. Thus, wherever the tolerizing antigen is present, local immunosuppression will occur.

By using E-selectin as the tolerizing agent, one can target immunosuppression to activated vessel segment.

### SUMMARY OF THE INVENTION

The present invention features include recombinant mammalian E-selectin peptides, nucleic acids encoding these peptides, vectors and cells having these nucleic acids, and methods of making the peptides. Further inventive features include methods of treating inflammatory diseases using recombinant mammalian E-selectin peptides to induce mucosal tolerance to E-selectin.

The invention provides a series of mammalian E-selectin peptides. One E-selectin peptide consists essentially of residues #20-303 of wild type human E selectin (SEQ ID NO: 1). This peptide may have one or more C terminal tags attached to it, including a carboxy terminal dipeptide RS. In addition the invention includes this peptide with an N terminal secretory signal peptide attached to it. While human recombinant peptides are preferred, other mammalian peptides, preferably from 200-400 amino acids, having at least 60% identity with SEQ ID NO: 1, may be used. Mixtures and combinations of mammalian E-selectin peptides are also contemplated.

The C terminal tags of the peptides of the invention include purification tags and stabilization tags such as c-myc tags and histidine tags. The N terminal secretory signal peptides include both mammalian and insect cell derived peptides. The N terminal secretory signal peptides include the AcMNPV gp64 env secretory sequence MGWSWIFLFLLSGTASVHS (SEQ ID NO:3), the signal peptide sequence MGWSWIFLFLLSGTAS (SEQ ID NO:4), as well as the wild type human signal sequence peptide of MIASQFLSALTLVLLIKESGA (SEQ ID NO:2). The peptides of the invention can be produced in various cell lines and include insect cells, mammalian cells, bacterial cells and yeast.

The invention also features nucleic acid molecules that encode a series of mammalian E-selectin peptides. The nucleic acid molecules encode a E-selectin peptide which consists essentially of residues #20-303 of wild type human E-selectin (SEQ ID NO: 1). The nucleic acid molecules encoding this E-selectin peptide which may have one or more C terminal tags attached to it, including a carboxy terminal dipeptide RS. In addition the invention includes nucleic acid molecules that encode this basic E-selectin peptide with an N terminal secretory signal peptide attached to it. While nucleic acid molecules that encode human recombinant peptides are preferred, preferably of 200-400 amino acids, encoding other mammalian peptides having at least 60% identity with SEQ ID NO: 1 may be used. Mixtures and combinations of mammalian E-selectin peptides are also contemplated.

The invention also features nucleic acid molecules that encode purification tags and stabilization tags such as c-myc tags and histidine tags. The nucleic acid molecules can encode N terminal secretory signal peptides including both mammalian and insect cell derived peptides. The nucleic acid molecules can encode the N terminal secretory signal peptides including the AcMNPV gp64 env secretory sequence MGWSWIFLFLLSGTASVHS (SEQ ID NO: 3), the signal peptide sequence MGWSWIFLFLLSGTAS (SEQ ID NO: 4), as well as the wild type human signal sequence peptide of MIASQFLSALTLVLLIKESGA (SEQ ID NO: 2). The nucleic acid molecules can be used to produce the peptides of the invention in various cell lines and include insect cells, mammalian cells, bacterial cells and yeast.

The invention also features a baculovirus having a nucleotide sequence encoding an E-selectin peptide, a vector having a nucleotide sequence encoding an E-selectin peptide, or a recombinant baculovirus transfer vector including the DNA segment encoding a baculovirus signal peptide linked to the nucleic acid encoding an E-selectin peptide. The DNA sequence is positioned so that the encoded E-selectin peptide is translated in frame with the encoded signal peptide. This recombinant baculovirus transfer vector is preferably operably linked to a baculovirus promoter to express the nucleic acid encoding an E-selectin peptide in a host cell. The host cells can include insect cells, bacterial cells and mammalian cells. Preferably, the recombinant baculovirus transfer vector includes sequences for secreting an E-selectin peptide of the invention into a culture medium for said insect host cell.

The invention also features a composition having one or more E-selectin peptides or nucleotides and a carrier, preferably a pharmaceutically acceptable carrier. Isolated cells and compositions of cells that include an E-selectin peptide or a nucleotide encoding an E-selectin peptide are also part of the invention. Useful cells include mammalian cells, bacterial cells and insect cells, preferably insect cells.

The invention further features a method of producing an E-selectin peptide of the invention. This includes starting by constructing a recombinant transfer vector which includes a DNA segment encoding a baculovirus signal peptide linked to a nucleic acid encoding an E-selectin peptide, so that the signal sequence and the nucleic acid encoding an E-selectin peptide are translated in frame. The DNA segment encoding a baculovirus signal peptide is operably linked to a baculovirus promoter for expressing and secreting an E-selectin peptide in insect cells. First insect cells are cotransfected with the recombinant transfer vector and baculovirus DNA to generate recombinant baculovirus. The recombinant baculovirus is harvested. Second insect cells are infected with the harvested recombinant baculovirus. The infected insect cells are cultured in a medium to express and secrete an E-selectin peptide. The culture medium is collected and purified to collect the E-selectin peptide.

The invention also includes a method of treating an inflammation mediated disease or condition in an individual, by inducing mucosal tolerance to a soluble E-selectin peptide. This is accomplished by administration of individual multiple low doses of E-selectin through a nasal or oral route of administration.

### BRIEF DESCRIPTION OF THE DRAWINGS

The objects and features of the invention can be better understood with reference to the following detailed description and drawings.

FIG. 1 (SEQ ID NO:8) is the predicted amino acid sequence and protein structure function aldesignations of a recombinant human E-selectin peptide.

FIG. 2 shows the alignment of recombinant E-selectin peptides to wild-type human E-selectin.

### DETAILED DESCRIPTION OF THE INVENTION

This invention provides recombinant E-selectin peptides, DNA encoding the E-selectin peptides, and methods of making and using the peptides. The following definitions are used throughout.

### Definitions

The practice of the present invention will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995). All patents, patent applications, and publications mentioned herein, both supra and infra, are hereby incorporated by reference in their entireties.

As used herein, the “N terminal” region of a peptide refers to the peptide sequences encoded by polynucleotide sequences (double-stranded or single-stranded) located within or at the 5′ end of a gene, and includes, but is not limited to, the 5′ protein coding region of a gene. As used herein, the “amino terminal” region refers to the amino terminal end of a peptide up to the first 300 amino acids or ⅓ of the peptide, starting at the first amino acid of the peptide. The “amino terminal” region of a peptide is not shorter than 3 amino acids in length and not longer than 350 amino acids in length. Other possible lengths of the “amino terminal” region of a peptide include but are not limited to 5, 10, 20, 25, 50, 100 and 200 amino acids.

As used herein, the “carboxy terminal” or “C terminal” region of a peptide refers to the polypeptide sequences encoded by polynucleotide sequences (double-stranded or single-stranded) located within or at the 3′ end of a gene, and includes, but is not limited to, the 3′ protein coding region of a gene. As used herein, the “carboxy terminal” region refers to the carboxy terminal end of a peptide up to 300 amino acids or ⅓ of the peptide from the last amino acid of the peptide. The “3′ end” does not include the polyA tail, if one is present. The “carboxy terminal” region of a polypeptide is not shorter than 3 amino acids in length and not longer than 350 amino acids in length. Other possible lengths of the “carboxy terminal” region of a peptide include, but are not limited to, 5, 10, 20, 25, 50, 100 and 200 amino acids.

An E-selectin peptide that has a similar amino acid sequence to a second E-selectin peptide is one that satisfies at least one of the following: (a) a E-selectin peptide having an amino acid sequence that is at least 60%, at least 65%, at least 70%, at least 75%, at least 80%, at least 85%, at least 90%, at least 95% or at least 99% identical to the amino acid sequence of a second E-selectin peptide; (b) an E-selectin peptide encoded by a nucleotide sequence that hybridizes under stringent conditions to a nucleotide sequence encoding a second proteinaceous agent of at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) an E-selectin peptide encoded by a nucleotide sequence that is at least 60%, at least 65%, at least 70%, at least 75%, at least 80%, at least 85%, at least 90%, at least 95% or at least 99% identical to the nucleotide sequence encoding a second E-selectin peptide.

A first E-selectin peptide with similar structure to a second E-selectin peptide refers to an E-selectin peptide that has a similar secondary, tertiary- or quaternary structure to the second E-selectin peptide. The structure of a E-selectin peptide can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). The two sequences may be the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of an E-selectin peptide analog refers to a second organic or inorganic molecule which possess a similar or identical function as E-selectin peptide and is structurally similar to E-selectin peptide. The term “analog” includes a molecule whose core structure is the same as, or closely resembles that of E-selectin peptide, but which has a chemical or physical modification. The term “analog” includes copolymers of E-selectin peptide that can be linked to other atoms or molecules. A “biologically active analog” and “analog” are used interchangeably herein to cover an organic or inorganic molecule that exhibits substantially the same agonist or antagonist effect of E-selectin peptide.

A “nucleotide analog” of E-selectin peptide, as used herein, refers to a nucleotide in which the pentose sugar and/or one or more of the phosphate esters is replaced with its respective analog. Exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., including any associated counterions, if present. Also included within the definition of “nucleotide analog” are nucleobase monomers which can be polymerized into polynucleotide analogs in which the DNA/RNA phosphate ester and/or sugar phosphate ester backbone is replaced with a different type of linkage. Further included within “nucleotide analogs” are nucleotides in which the nucleobase moiety is non-conventional, i.e., differs from one of G, A, T, U or C. Generally a non-conventional nucleobase will have the capacity to form hydrogen bonds with at least one nucleobase moiety present on an adjacent counter-directional polynucleotide strand or provide a non-interacting, non-interfering base.

As used herein, the term “effective amount” refers to the amount of a an E-selectin peptide or nucleic acid which is sufficient to reduce or ameliorate the progression, severity and/or duration of inflammation or one or more symptoms thereof, prevent the development of inflammation or one or more symptoms thereof, prevent the advancement of inflammation or one or more symptoms thereof, or enhance or improve the prophylactic or therapeutic effect(s) of another therapy.

As used herein, the term “effective amount” refers the amount of E-selectin peptide which is sufficient to induce tolerance to E-selectin through nasal administration.

As used herein, the term “fragment” in the context of a an E-selectin protein refers to a peptide or polypeptide comprising an amino acid sequence of at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a mammalian E-selectin. A fragment of a protein or polypeptide useful in the invention retains at least one function of a mammalian E-selectin. A fragment of a protein or polypeptide may retain two, three, four or more functions of a mammalian E-selectin.

As used herein, the term “in combination” when referring to therapeutic treatments refers to the use of more than one type of therapy (e.g., more than one prophylactic agent and/or therapeutic agent). The use of the term “in combination” does not restrict the order in which therapies (e.g., prophylactic and/or therapeutic agents) are administered to a subject. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours/, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second prophylactic or therapeutic agent) to a subject.

As used herein, “isolated” or “purified” when used in reference to a peptide or nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide or peptide present, but that it is essentially free (about 90-95% pure) of non-nucleotide or non-peptide material naturally associated with it, and thus is distinguished from isolated chromosomes.

As used herein, the terms “isolated” and “purified” in the context of a proteinaceous agent (e.g., a peptide, polypeptide, protein or antibody) refer to a proteinaceous agent which is substantially free of cellular material and in some embodiments, substantially free of heterologous proteinaceous agents (i.e., contaminating proteins) from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous proteinaceous agent (e.g., protein, polypeptide, peptide, or antibody; also referred to as a “contaminating protein”). When the proteinaceous agent is recombinantly produced, it is also preferably substantially free of culture medium, i.e., culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. Preferably, proteinaceous agents disclosed herein are isolated.

As used herein, “nucleic acid(s)” is interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double-stranded RNA or DNA or any combinations thereof.

As used herein, “nucleic acid” encompasses double-stranded DNA, single-stranded DNA and double-stranded or single-stranded RNA of more than 8 nucleotides in length. The term “polynucleotide” includes a polymeric form of nucleotides of any length, either ribonucleotides or deoxyribonucleotides, that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components.

As used herein, “patient” or “individual” refers to a mammal, preferably human, who is administered the E-selectin peptide.

As used herein, the phrase “pharmaceutically acceptable carrier” includes, but is not limited to, aqueous or nonaqueous compositions comprising salts of acidic or basic groups that may be present in compounds identified using the methods of the present invention. Compounds that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein, “polypeptide sequences encoded by” refers to the amino acid sequences obtained after translation of the protein coding region of a gene, as defined herein.

As used herein, the terms “protein” and “peptide” and “polypeptide” are used interchangeably to refer to a chain of amino acids linked together by peptide bonds. In a specific embodiment, a protein is composed of less than 200, less than 175, less than 150, less than 125, less than 100, less than 50, less than 45, less than 40, less than 35, less than 30, less than 25, less than 20, less than 15, less than 10, or less than 5 amino acids linked together by peptide bonds. A protein is composed of at least 200, at least 250, at least 300, at least 350, at least 400, at least 450, at least 500 or more amino acids linked together by peptide bonds.

A “protein coding region” refers to the portion of the mRNA encoding a polypeptide.

The present invention is based, in part, on the recognition that certain portions or domains of the extracellular domain of E-selectin may have beneficial effects. Recombinant peptides, preferably the 20-303 fragment of the human E-selectin peptide, and DNA and cells that encode or make these peptides are useful in this invention.

Example of a Composition Comprising Soluble E-Selectin Peptide Designed to Induce Tolerance

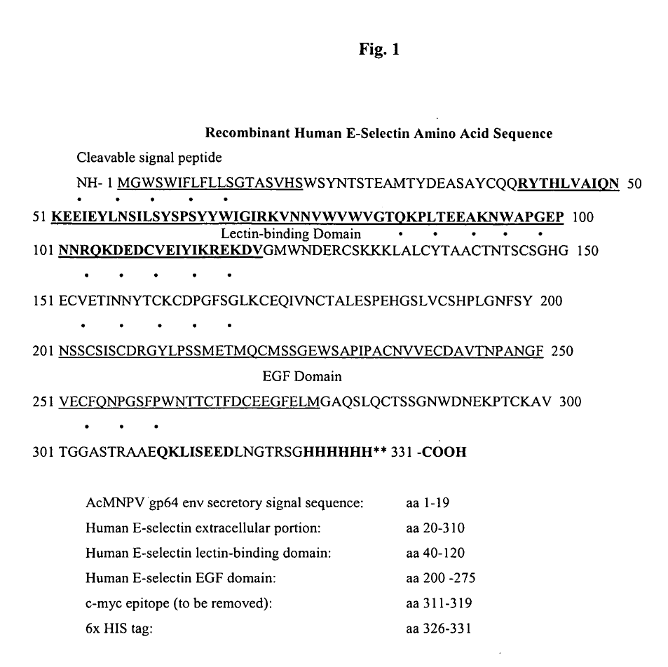

The E-selectin peptide is a clear soluble liquid protein solution that is provided in phosphate-buffered saline (PBS) solution. The drug substance is derived from Sf-9S insect cells (Spodoptera frugzperda from the Lepidopteran family) infected with recombinant AcMNPV baculovirus vector (Autographica californica multinuclear polyhedrosis virus from the Baculoviridae family) encoding at the extracellular portion of the human E-selectin protein with the lectin-binding epidermal growth factor (EDF) domains fused to gp64 secretory signal at the amino terminus and c-myc and polyhistidine peptide tags at the carboxy terminus (FIG. 1).

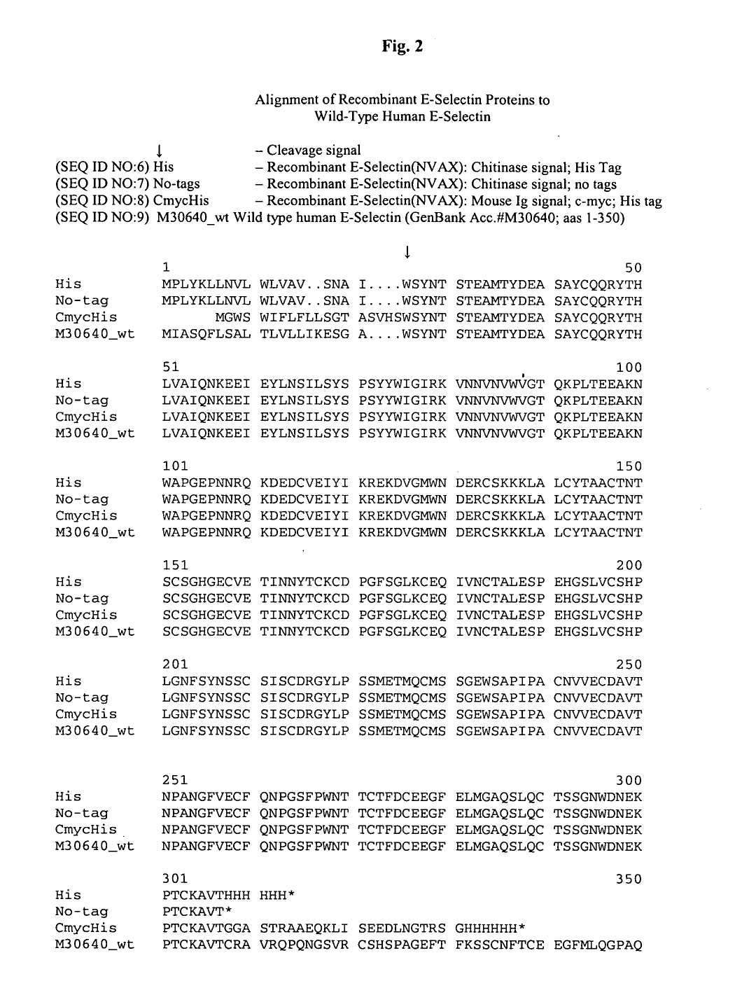

The cloned gene encoding the recombinant human E-selectin protein is a 331 amino acid polypeptide comprised of 19 amino acids of AcIVfNPV baculovirus gp64 envelope protein secretory signal peptide, 291 amino acids of the human E-selectin protein (aa 20-aa 310 extracellular portion with aa 40-aa 120 lectin-binding domain and aa 200—an 275 EGF domain), 9 amino acids of the c-myc protein, 6 amino acids of a neutral spacer peptide, and a 6 amino acid polyhistidine tag (6×HIS). In one alternative form, the c-myc tag is omitted, and in another alternative form, both the c-myc tag and the His tag are omitted, (FIG. 2). The secretory signal peptide, which is derived from the AcMINPV baculovirus gp64 envelope protein, facilitates intracellular transport of the target peptide, processing, and secretion of the recombinant human E-selectin protein. The human E-selectin extracellular polypeptide portion of the drug substance serves as a tolerogen effector molecule to stimulate suppression of inflammatory and other immune responses active in stroke pathology. The c-myc peptide, which is a monoclonal antibody epitope localized to the nontransforming domain of c-myc, acts an identity tag for the recombinant human E-selectin protein molecule during protein purification. The 6×HIS peptide, which binds to heavy metals such nickel, cobalt, and others with a strong binding constant (K_{d}>10⁻⁹ M), is used to purify recombinant human E-selectin proteins by immobilized metal affinity chromatography.

Virus Stocks Encoding an E-Selectin Peptide

Recombinant DNA cloning of a DNA fragment synthesized in vitro used a codon-optimized gene encoding the extracellular portion of the human E-selectin (amino acid residues 20-310) fused with carboxy terminal c-myc peptide and polyhistidine (6×HIS) peptide tags, based on nucleotide sequences available from GenBank Accession No. NM 000655, for expression in a baculovirus expression vector system (BEVS). A 999 bp Eco R I DNA fragment containing the human E-selectin gene was cloned initially in multiple cloning site of the subcloning vector pCR-Blunt II-TOPO (InVitrogen) and subsequently downstream of the polyhedron promoter within the polyhedra locus of the bacmid transfer vector, pFASTBACI (InVitrogen). Sf-9S insect cells were transfected with recombinant bacmid DNAs containing the human E-selectin gene within the AcMNPV genome. Recombinant baculoviruses were isolated from transfected cells and selected by plaque purification for viral clones expressing high levels of recombinant human E-selectin protein.

A master virus stock (9.6 L) was established by infection of Sf-9S insect cells (passage 60; WCB #38) at a MOI of 0.1 pfu/ml from an amplified plaque-purified recombinant baculovirus isolate (R612) that expressed high levels of recombinant human E-selectin protein and afforded high titers of virus. The master virus stock was characterized for gene integrity and recombinant protein production. The characterization which included microbial sterility assays, mycoplasma detection assay, spiroplasma detection assay, LAL endotoxin chromogenic assay, in vitro adventitious agent testing, and in vivo adventitious agent testing (AnMed/Taconic), was performed on samples of the master virus stock. Adventitious agent tests were able to detect the presence of RNA viruses that may infect insect cells or that may be carrier virus.

The identity of the recombinant human E-selectin gene sequences fused to c-myc and polyhistidine peptide tags was demonstrated by nucleotide sequence determination and analysis of both DNA strands of insert and flanking nucleotide sequences from baculovirus genomic DNA isolated from the recombinant baculoviruses in the master virus stock and encoding the human E-selectin gene. Nucleotide sequence analysis revealed a 100% match of the human E-selectin gene sequences in the genomic DNA from the master virus stock and in the baculovirus transfer vector, pFASTBAC 1, synthesized in vitro and used in the cloning of the gene. The amino acid sequence predicted from the nucleotide sequence from the genomic DNA sample matched 100% with the predicted amino acid sequence of human E-selectin. The master virus stock passed the identity testing.

The ability of the master virus stock to support virus replication at high titers was determined by baculovirus plaque assays of clarified supernatants from Sf-9S insect cells infected for three (3) days at a multiplicity of infection (MOI) of 0.1 plaque forming units (pfu) per cell. A virus titer of 5×10⁷ pfu/ml was determined by the baculovirus agarose plaque assay in Sf-9S insect cells using a sample of the master virus stock passaged in Sf9S insect cells. The master virus stock was evaluated further for recombinant human E-selectin protein production by SDS-PAGE and Western blot analyses of cell lysates and supernatants from Sf-9S insect cells infected at days 1 to 3 days with the master virus stock at a MOI of 3-5 pfu per cell. Cell lysates and cell supernatants contained a recombinant protein with a molecular weight of 50 kDa and with specific binding to a monoclonal antibody (BBA2; R&D) to human E-selectin protein.

The master virus stock, which was qualified for production of virus stocks, was used in the production of working virus stocks destined for manufacturing of recombinant human E-selectin protein products. Working virus stock (9.6 L) was established by infection of Sf-9S insect cells (passage 52; WCB#37) at a MOI of 0.1 pfu/cell with an inoculum of the master virus stock. Master and working virus stocks were stored in light-protective wrapped PETG bottles short term (<3 months) in a light-protected cold box (2-8° C.) and long term in ultralow freezers at ≦70° C.

### Cell Banks

Expression of recombinant human E-selectin protein was realized best in Sf-9S insect cells. A master cell bank of Sf-9S insect cells was established from a single vial of the Sf-9S cells, which were adapted to serum-free media, suspension cell culture, and selected for secretion of recombinant proteins expressed from baculovirus vectors from parental Sf-9 cells obtained from the ATCC. The Sf-9S master cell bank was established with a Sf-9 cell line derived originally from Spodoptera frugiperda ovarian epithelial cells but has undergone several significant adaptations to maximize recombinant protein expression in large scale bioreactors in serum-free media as suspension cultures.

Sf-9S master cell bank consists of 586×3.5 ml cryovials of Spodoptera frugiperda cells at cell passage no. 48 in insect cell freezing media (7.5% dimethyl sulfoxide, 46% Sf-900 II SFM, 47% conditioned media). The working cell bank was established by thawing a cryovial (3.5×10⁷ cells total) from the master cell bank and seeding cells into fresh HyQ SFX serum-free insect cell media (100 ml; lot no. ALF 14050) in a shaker flask (500 ml). The cells were allowed to acclimate for several days and grow as a suspension culture at 28° C. and 125 rpm. When the cell density reached 0.6×10⁷ cells/ml, the culture was divided at a split ratio of 1:20 into more shaker flasks at a final volume of 800 ml per 2 L flask. The new cultures were subcultured similarly for several passages to ensure that the cells were growing optimally and were not contaminated. The Sf-9S cell culture at passage 49 reached a cell density of 5.36×10⁶ cells/ml and a viability of 94%, and the cells were isolated by low-speed centrifugation (500×g) and resuspended in insect cell freezing media, comprised of the following:

46.5 parts HyQ SFX serum-free insect cell medium (conditioned)
46.5 parts HyQ SFX serum-free insect cell medium (fresh)
7.0 parts Dimethyl Sulfoxide (Sigma lot no. 68H1092)

The cells (1×10⁷ cells/ml) were dispensed aseptically into 49 cryovials (3.5 ml/vial) and 30 cryovials (1.0 ml/vial) and were frozen slowly at 1° C. per minute for storage in an ultralow freezer at <−70° C.

Nucleotide Sequence of Codon-Optimized Recombinant Human E-Selectin

The following sequence is a nucleotide sequence of codon-optimized recombinant human E-selectin gene from baculovirus genome in master virus stock for production of recombinant human E-selectin protein.

(SEQ D NO: 5)

| | |
|---|---|
| 1 | ATGGGTTGGTCTTGGATTTTCTTGTTCTTGTTGTCTGGTACTGCTTC |
| | TGT |
| 51 | TCACTCTTGGTCTTACAACACTTCTACTGAAGCTATGACTTACGACG |
| | AAG |
| 101 | CTTCTGCTTACTGTCAACAAAGATACACTCACTTGGTTGCTATTCAA |
| | AAC |
| 151 | AAGGAAGAAATTGAATACTTGAACTCTATTTTGTCTTACTCTCCATC |
| | TTA |
| 201 | CTACTGGATTGGTATTAGAAAGGTTAACAACGTTTGGGTTTGGGTTG |
| | GTA |
| 251 | CTCAAAAGCCATTGACTGAAGAAGCTAAGAACTGGGCTCCAGGTGAA |
| | CCA |
| 301 | AACAACAGACAAAaGGACGAAGACTGTGTTGAAATTTACATTAAGAG |
| | AGA |
| 351 | AAAGGACGTTGGTATGTGGAACGACGAAAGATGTTCTAAGAAGAAGT |
| | TG |
| 401 | CTTTGTGTTACACTGCTGCTTGTACTAACACTTCTTGTTCTGGTCAC |
| | GGT |
| 451 | GAATGTGTTGAAACTATTAACAACTACACTTGTAAGTGTGACCCAGG |
| | TTT |
| 501 | CTCTGGTTTGAAGTGTGAACAAATTGTTAACTGTACTGCTTTGGAAT |
| | CTC |
| 551 | CAGAACACGGTTCTTTGGTTTGTTCTCACCCATTGGGTAACTTCTCT |
| | TAC |
| 601 | AACTCTTCTTGTTCTATTTCTTGTGACAGAGGTTACTTGCCATCTTC |
| | TAT |
| 651 | GGAAACTATGCAATGTATGTCTTCTGGTGAATGGTCTGCTCCAATTC |
| | CAG |
| 701 | CTTGTAACGTTGTTGAATGTGACGCTGTTACTAACCCAGCTAACGGT |
| | TTC |
| 751 | GTTGAATGTTTCCAAAACCCAGGTTCTTTCCCATGGAACACTACTTG |
| | TAC |
| 801 | TTTCGACTGTGAAGAAGGTTTCGAATTGATGGGTGCTCAATCTTTGC |
| | AAT |
| 851 | GTACTTCTTCTGGTAACTGGGACAACGAAAAGCCAACTTGTAAGGCT |
| | GTT |
| 901 | ACTGGTGGTGCTTCTACTAGAGCTGCTGAACAAAAGTTGATTTCTGA |
| | AGA |
| 951 | AGACTTGAACGGTACTAGATCTGGT |

Cell Amplification

Cell amplification of cells containing recombinant human E-selectin was comprised of 16.8 liters in 2.0 L Corning plastic shaker flasks (21 flasks containing 800 ml of HyQ SFX serum-free media per flask). Culture flasks were incubated in a platform shaker incubator (Fisher) equipped with spring-loaded flask clamps. Cells were incubated at 28+/−1° C. and 125+/−25 rpm.

Virus Infection

The Sf-9S cells were diluted with fresh serum-free media to a final cell density of 2.0×10⁶ cells/ml and distributed as 800 ml aliquots into 21 flasks (2 L). The insect cells were infected with baculovirus containing HuE-selectin peptide at a MOI of 3 pfu/cell. Virus was retrieved from the virus stock and dispensed into flasks in a Class 100 biosafety hood. The infected cell cultures were maintained at 28° C. and 125 rpm. The infected cell cultures were monitored periodically for viral cytopathic effects (CPE), cell density, and cell viability. The virus infection was carried out for 3 days.

At 3 days post-infection, the viral CPE reached +3 (i.e., inclusion body formation, and membrane ruffling), cell density was 1.1×10⁶ cells/ml, and cell viability decreased to 50%. The infected cell cultures were harvested as described below.

Harvest

Infected cell suspensions were transferred from flasks to 500 ml centrifuge bottles in a biosafety cabinet. Infected cell suspensions were subjected to low-speed centrifugation in a Sorval RC-5B centrifuge at 2300 rpm and 4° C. for 10 mm. to remove infected cells. The infected cell culture supernatants containing extracellular recombinant human E-selectin were clarified by centrifugation in a Sorval RC-5B centrifuge at 7500 rpm and 4° C. for 45 mm. Clarified supernatants were decanted into a 20 liter glass carboy within a class 100 biosafety hood and stored overnight in a cold box at 2-8° C. for subsequent concentration and diafiltration.

Concentration and Diafiltration by Ultrafiltration

The clarified cell culture supernatant (16.0 L) containing extracellular recombinant human E-selectin peptides was concentrated using an A/G Technologies Flex StandBenchtop Pilot ultrafiltration system in order to obtain a manageable volume for further purification. With this system, the clarified cell culture supernatant was transferred at a flow rate of 230 ml/mm. through sanitized silicone tubing with a Masterfiex peristaltic pump from a 20 L glass carboy to a A/G Tech UFP-1,0-C-9A hollow fiber ultrafiltration cartridge, which had a molecular weight cutoff (MWCO) of 10 kDa. The retentate containing recombinant human E-selectin peptide was collected separately from the filtrate and concentrated (20-fold) to 0.8 L by continuous passage though the spiral wound ultrafiltration cartridge. After concentration, the concentrated cell culture supernatant was diafiltered for 90 mm. with 10 L of Q buffer 1. The concentrated diafiltrate (0.8 L) and two rinses (0.7 L each) of the cartridge were collected into sterile Nalgene bottles (3.2 L total) and stored in a cold box (2-8° C.) overnight for subsequent protein purification by Q anion exchange chromatography.

Q Sepharose Anion Exchange Chromatography

The initial protein capture step in downstream processing of recombinant human E-selectin peptide drug substance was an anion exchange chromatography step using a strong anion exchange resin, Q Sepharose Fast Flow. This step was intended to remove endotoxins, process excipients, and host protein contaminants away from recombinant human E-selectin peptides. The Q anion exchange chromatography step was performed using a validated Pharmacia AKTA Explorer Biopilot FPLC system controlled by Unicorn® software. Q Sepharose Fast Flow resin (400 ml) was loaded into a Pharmacia XX 50/30 chromatographic column. The Q column was sanitized and regenerated with Q regeneration buffer [0.5 N sodium hydroxide and 1.0 M sodium chloride] at a flow rate of 10 ml/min., rinsed with 5 column volumes (2000 ml) of WFI water at a flow rate of 10 ml/min to a pH of 7.1, and equilibrated with five column volumes (2000 ml) of Q buffer 1 at a flow rate of 10 ml/mm.

The concentrated diafiltrate (3.2 L) was loaded at a flow rate of 20 ml/min. (13.6 mg of protein/ml Q resin). The loaded Q column washed with 10 column volumes (4000 ml) of Q buffer 1 at a flow rate of 20 ml/min. Q column flow through (FT; 3200 ml) and wash (1600 ml) fractions were collected and stored at 4° C. for in-process testing for residual unbound recombinant human E-selectin peptides. Proteins bound to the Q column were eluted at a flow rate of 20 ml/min with Q buffer 2 forming a linear 0-1000 mM linear gradient of sodium chloride. Fractions (200×10 ml) of the UV₂₈₀ absorbent Q eluate material were collected and stored temporarily in a cold box at 4° C. The used Q column was sanitized with Q regeneration buffer.

Samples (0.1 ml) of Q eluate fractions, as well as load, flow through, and wash fractions, were subjected to in-process testing including SDS-PAGE and Western blot analyses using a human E-selectin sera. Q eluate fractions containing recombinant human E-selectin proteins as the major constituent were identified in a single peak (fractions 80-112) by SDS-PAGE and Western blot analyses and pooled (320 ml). No significant amounts of recombinant human E-selectin proteins failed to bind to the Q column; thus, no reprocessing of the FT fractions was necessary.

Ni-NTA Agarose Affinity Chromatography

The presence of a polyhistidine (6×HIS) tag peptide at the carboxyl terminus of the recombinant human E-selectin protein permitted the purification of these heavy metal binding proteins from other proteins remaining in the Q pooled eluate fraction by immobilized metal affinity chromatography using a Ni⁺⁺ based resin. The Ni-NTA affinity chromatographic step in the downstream manufacturing process was used to purify recombinant human E-selectin protein and remove remaining host protein contaminants and baculoviruses. The Ni-NTA affinity chromatography step was performed using a validated Pharmacia AKTA Explorer Biopilot FPLC system controlled by Unicorn® software. Ni-NTA Agarose Superfiow resin (38 ml) was loaded into a Pharmacia XK 26 chromatographic column. The Ni-NTA was charged with nickel sulfate hexahydrate (0.1 M), sanitized with 0.5 N NaOH at a flow rate of 3 ml/min., rinsed with 5 column volumes of WFI water at a flow rate of 3 ml/min., and equilibrated with five column volumes (190 ml) of Ni-NTA buffer 1 at a flow rate of 3 ml/min. to a pH of 8.5. The Q polled eluate fraction was loaded at a flow rate of 3 ml/min. (19.8 protein/ml of Ni-NTA resin). The loaded Ni-NTA column washed with 3 column volumes (115 ml) of Ni-NTA buffer I at a flow rate of 3 ml/min. Ni-NTA column FT (320 ml) and wash (115 ml) fractions were collected and stored at 4° C. for in-process testing for residual unbound recombinant human E-selectin proteins. Proteins bound to the Ni-NTA column were eluted at a flow rate of 3 ml/mm with Ni-NTA buffer 2 forming a linear 0-300 mM linear gradient of sodium imidazole. Fractions (43×3 ml) of the DY₂₈₀ absorbent Ni-NTA eluate material were collected and stored temporarily in a cold box at 4° C. The used Ni-NTA column was sanitized with EDTA regeneration buffer.

Samples (0.1 ml) of Ni-NTA eluate fractions, as well as load, flow through, and wash fractions, were subjected to in-process testing including SDS-PAGE and Western blot analyses using a human E-selectin sera. Ni-NTA eluate fractions containing recombinant human E-selectin proteins as the major constituent were identified in a single peak (fractions 12-26) by SDS-PAGE and Western blot analyses and pooled (43 ml). No significant amounts of recombinant human E-selectin proteins failed to bind to the NiNTA column; thus, no reprocessing of the FT fractions was necessary.

Sample (2 ml) of the pooled Ni-NTA eluate column fractions were subjected to in-process testing including SDS-PAGE and Western blot analyses using a human E-selectin sera, BCA protein assay, and LAL endotoxin assay. Additionally, a baculovirus agarose plaque assay was performed on an aliquot of the Ni-NTA polled eluate fraction to enumerate the amount of baculovirus present at this stage of the purification process. The virus titer was 6.42×10⁷ pfu/ml for a total of 2.76×10⁹ pfu for a 3 log₁₀ reduction in virus afforded by Q and Ni-NTA chromatographic steps.

Diafiltration

To remove imidazole, a process excipient, from the Ni-NTA pooled eluate fraction and formulate the drug substance in the appropriate buffer, PBS solution, the Ni-NTA pooled eluate fraction was subjected to diafiltration in a cold box (2-8° C.). The pooled Ni-NTA eluate fraction was dialyzed against 2×90 volumes (4 L) of PBS solution for 15 and 7 hours, respectively, at 22° C. The final dialysate volume was 41 ml. Sample (1 ml) of dialysate was removed for in-process testing including SDS PAGE analysis, Western blot analysis, BCA protein assay, and LAL kinetic chromogenic assay.

Terminal Filtration

To remove microbial contaminants, the dialysate (41 ml) was passed aseptically in a biosafety hood (class 100) through a 0.22˜i Millipore Stericap filter membrane into a sterile Nalgene bottle. The used membrane was subjected to a bubble point assay to determine membrane integrity, the result (50 psi) exceeded the integrity membrane specification of 32 psi and provided assurance for microbial clearance from the drug substance. The final volume of the filtrate was 36.5 ml. The 0.2μ filtrate was stored in an ultralow freezer at <−70° C. The 0.2μ filtrate was thawed, diluted with PBS solution to a final volume of 95 ml to prevent protein aggregation at the previously high protein concentration, and filtered aseptically through a second 0.2μ membrane in a biosafety hood (class 100). The results of bubble point testing of the second used 0.2μ membrane indicated that the membrane was intact.

Samples of the first 0.2μ filtrate were subjected to BCA protein and LAL in-process testing. The result of the BCA protein assay for the first 0.2μ filtrate was 5.28 mg/ml for a total yield of 192.72 mg. The result of the LAL endotoxin assay for the first 0.2μ filtrate was 1.84 EU/ml for a total of 67 EU.

A total volume of 95 ml was realized from the second terminal filtration. To remove residual baculoviruses in the drug substance, a Pall DV2O sub 0.1μ membrane filter cartridge was utilized in the formulation and filtration step of the drug products. The total endotoxin load for the final bulk product (drug substance) was 45.6 EU; the total protein yield for the final bulk product was 133 mg, as determined by a validated BCS protein assay.

Delayed Type Hypersensitivity Assay

Delayed type hypersensitivity tests were performed in hypertensive rats following intranasal treatment with various doses of recombinant human E-selectin. Delayed type hypersensitivity assay of drug substance samples (1.0 ml) was performed to determine in vivo product potency, which is correlated with the ability of human E-selectin to tolenze and prevent stroke in hypertensive animals. DTH suppression in this study involved the measurement of animal ear thickness caused by inflammation as a function of different doses of recombinant human E-selectin and placebo used in the induction of mucosal tolerance.

Spontaneously hypertensive stroke-prone (SRR-SP) rats (n=20) were divided into four groups and inoculated intranasally (20 μl/nare) every other day for five (5) treatments:

Group 1: PBS placebo, tolerized with 40 μl treatments, n=5,
Group 2: recombinant human E-selectin 5 V μg/40 μl treatments, n=5
Group 3: recombinant human E-selectin μl/40 μl treatments, n=5
Group 4: recombinant human E-selectin 0.1 μg/40 μl treatment, n=5

Two weeks after the end of the tolerization schedule, the animals were immunized (antigen sensitization) subcutaneously with aliquots (200 μl) of recombinant human E-Selectin formulated with complete Freund's adjuvant (FCA) at a final antigen concentration of 375 μg/ml. Two weeks after immunization, the ear thickness of treated animals was measured using standard skin-fold calipers. Afterwards ear-lobe injections (100 p.l) of recombinant human E-selectin at an antigen concentration of 50 μg/100 μl in PBS (re-application of antigen or antigen challenge).

Repeat ear thickness measurements were done at 48 and 72 hours post-challenge to assess the delayed-type hypersensitivity.

Tolerization of lymphocytes to E-selectin, in particular mucosal tolerization, is an effective method of treatment of inflammatory diseases including:

Elevated levels of proinflammatory cytokines are also associated with a number of diseases and conditions, including autoimmune diseases. Inflammation associated diseases include, but are not limited to, toxic shock syndrome, rheumatoid arthritis, osteoarthritis, diabetes and inflammatory bowel disease, dementia associated with HIV infection, glaucoma, optic-neuropathy, optic neuritis, retinal ischemia, laser induced optic damage, surgery or trauma-induced proliferative vitreoretinopathy, cerebral ischemia, hypoxia-ischemia, hypoglycemia, domoic acid poisoning, anoxia, carbon monoxide or manganese or cyanide poisoning, Huntington's disease, Alzheimer's disease, Parkinson's disease, meningitis, multiple sclerosis and other demyelinating diseases, amyotrophic lateral sclerosis, head and spinal cord trauma, seizures, convulsions, olivopontocerebellar atrophy, neuropathic pain syndromes, diabetic neuropathy, HIV-related neuropathy, MERRF and MELAS syndromes, Leber's disease, Wemicke's encephalophathy, Rett syndrome, homocysteinuria, hyperprolinemia, hyperhomocysteinemia, nonketotic hyperglycemia, hydroxybutyric aminoacidurias, sulfite oxidase deficiency, combined systems disease, lead encephalopathy, Tourett's syndrome, hepatic encephalopathy, drug addiction, drug tolerance, drug dependency, depression, anxiety and schizophrenia, traumatic arthritis, Guillain-Barre syndrome, Crohn's disease, ulcerative colitis, psoriasis, graft versus host disease, systemic lupus erythematosus, glomerulonephritis, reperfusion injury, sepsis, bone resorption diseases including osteoporosis, chronic obstructive pulmonary disease, congestive heart failure, atherosclerosis, toxic shock syndrome, asthma, contact dermatitis, percutaneous transluminal coronary angioplasty (PTCA) and insulin-dependent diabetes mellitus.

Variations, modifications, and other implementations of what is described herein will occur to those of ordinary skill in the art without departing from the spirit and scope of the invention. The references provided below are incorporated herein by reference in their entireties. All patents, patent applications, and published references cited herein are hereby incorporated by reference in their entirety.

While this invention has been particularly shown and described with references to preferred embodiments thereof, it will be understood by those skilled in the art that various changes in form and details may be made therein without departing from the scope of the invention encompassed by the appended claims. Those skilled in the art will recognize that other embodiments and configurations known in the art would be within the spirit and scope of the present invention.

What is claimed is:

1.-25. (canceled)

26. An isolated E-selectin polypeptide consisting of amino acid residues 20 to 301 of SEQ ID NO: 7.

27. The polypeptide of claim 26, wherein said polypeptide is attached to an N-terminal secretory signal peptide.

28. The polypeptide of claim 27, wherein said N-terminal secretory signal peptide is selected from the group consisting of SEQ ID NO: 3 and SEQ ID NO: 4.

29. The polypeptide of claim 26, wherein said polypeptide is produced in an insect cell.

30. A nucleic acid encoding a polypeptide consisting of amino acid residues 20 to 301 of SEQ ID NO: 7.

31. The nucleic acid of claim 30, wherein said nucleic acid is attached to a nucleic acid encoding an N-terminal secretory signal peptide.

32. The nucleic acid of claim 31, wherein said N-terminal secretory signal peptide is selected from the group consisting of SEQ ID NO: 3 and SEQ ID NO: 4.

33. A baculovirus comprising a nucleotide sequence encoding a polypeptide of claim 26.

34. A vector comprising a nucleotide sequence encoding a polypeptide of claim 26.

35. A recombinant baculovirus vector comprising the DNA segment encoding a baculovirus signal peptide linked to the nucleic acid encoding the polypeptide of claim 26, said nucleic acid being translationally in frame with the DNA segment encoding said signal peptide.

36. The recombinant baculovirus transfer vector of claim 35, operably linked to a baculovirus promoter to form an operable linkage for expressing said nucleic acid encoding the polypeptide of claim 26 in an insect host cell.

37. The recombinant baculovirus transfer vector of claim 36, which includes sequences for secreting the polypeptide of claim 26 into a culture medium for said insect host cell.

38. An isolated cell comprising the polypeptide of claim 26.

39. An isolated cell comprising the nucleic acid of claim 30.

40. The isolated cell of claim 39, wherein said cell is selected from the group consisting of a mammalian cell, a bacterial cell, and an insect cell.

41. The isolated cell of claim 40, wherein said cell is an insect cell.

42. A method of producing a polypeptide according to claim 26, comprising the steps of:
a) constructing a recombinant transfer vector which comprises a DNA segment encoding a baculovirus signal peptide linked to the nucleic acid of claim 30, said nucleic acid translationally in frame with the DNA segment encoding said signal peptide and operably linked to a baculovirus promoter for expressing a polypeptide of claim 26 in insect cells and secreting said polypeptide;
b) co-transfecting first insect cells with the recombinant transfer vector and baculovirus DNA to generate recombinant baculovirus;
c) harvesting the recombinant baculovirus;
d) infecting second insect cells with the harvested recombinant baculovirus and culturing the infected insect cells in a culture medium to express and secrete the polypeptide of claim 26,
and e) collecting the culture medium and purifying the secreted polypeptide of claim 26.

43. A method of treating an inflammation mediated disease or condition in an individual in need thereof by inducing mucosal tolerance to a soluble E-selectin polypeptide, comprising administering to said individual multiple low doses of E-selectin through nasal administration, wherein said E-selectin consists of the polypeptide of claim 26.

44. A composition comprising a polypeptide of claim 26 and a carrier.

45. A composition comprising the nucleic acid of claim 30 and a carrier.


SEQUENCE LISTING (9 sequences)

SEQ ID NO: 1 - protein, 610 aa, Homo sapiens
  misc_feature: Wild type human E-selectin
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 2 - protein, 21 aa, Artificial Sequence
  WILD TYPE HUMAN SIGNAL SEQUENCE PEPTIDE
MIASQFLSALTLVLLIKESGA

SEQ ID NO: 3 - protein, 19 aa, Artificial Sequence
  AcMNPV gp64 env secretory sequence
MGWSWIFLFLLSGTASVHS

SEQ ID NO: 4 - protein, 16 aa, Artificial Sequence
  Signal Peptide Sequence
MGWSWIFLFLLSGTAS

SEQ ID NO: 5 - DNA, 974 bp, Artificial Sequence
  Nucleotide Sequence of Codon-Optimized Recombinant HumanE-selectin Gene
atgggttggtcttggattttcttgttcttgttgtctggtactgcttctgttcactcttggtcttacaacacttctactgaagctatgacttacgacgaagcttctgcttactgtcaacaaagatacactcacttggttgctattcaaaacaaggaagaaattgaatacttgaactctattttgtcttactctccatcttactactggattggtattagaaaggttaacaacgtttgggtttgggttggtactcaaaagccattgactgaagaagctaagaactgggctccaggtgaaccaaacaacagacaaaaggacgaagactgtgttgaaatttacattaagagagaaaaggacgttggtatgtggaacgacgaaagatgttctaagaagaagttgctttgtgttacactgctgcttgtactaacacttcttgttctgctcacggtgaatgtgttgaaactattaacaactacacttgtaagtgtgacccaggtttctctggtttgaagtgtgaacaaattgttaactgtactgctttggaatctccagaacacggttctttggtttgttctcacccattgggtaacttctcttacaactcttcttgttctatttcttgtgacagaggttacttgccatcttctatggaaactatgcaatgtatgtcttctggtgaatggtctgctccaattccagcttgtaacgttgttgaatgtgacgctgttactaacccagctaacggtttcgttgaatgtttccaaaacccaggttctttcccatggaacactacttgtactttcgactgtgaagaaggtttcgaattgatgggtgctcaatctttgcaatgtacttcttctggtaactgggacaacgaaaagccaacttgtaaggctgttactggtggtgcttctactagagctgctgaacaaaagttgatttctgaagaagacttgaacggtactagatctggt

SEQ ID NO: 6 - protein, 307 aa, Artificial Sequence
  Recombinant E-Selectin Chitinase Signal, His Tag
MPLYKLLNVLWLVAVSNAIWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTHHHHHH

SEQ ID NO: 7 - protein, 301 aa, Artificial Sequence
  Recombinant E-selectin; Chitinase signal, no tags
MPLYKLLNVLWLVAVSNAIWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVT

SEQ ID NO: 8 - protein, 331 aa, Artificial Sequence
  Recombinant E-Selectin; Mouse Ig Signal, c-myc, His tag
MGWSWIFLFLLSGTASVHSWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTGGASTRAAEQKLISEEDLNGTRSGHHHHHH

SEQ ID NO: 9 - protein, 346 aa, Artificial Sequence
  Wild type Human E-selectin (GenBank Accession No. #M30640, amino acids 1-350)
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQ